(19) United States
(12) Patent Application Publication
(10) Pub. No.: US 2023/0332134 A1
(43) Pub. Date: Oct. 19, 2023

(54) AUTOMATED INTEGRATED SYSTEM FOR CONTINUOUS COMMERCIAL-SCALE PLASMID PRODUCTION

(71) Applicants: Nanjing GenScript Biotech Co., Ltd., Nanjing (CN); GenScript USA Inc., Redmond, WA (US)

(72) Inventors: Stephen Richard Hughes, Piscataway, NJ (US); Xiaoli Fu, Piscataway, NJ (US); Yifan Li, Nanjing (CN); Yuzhuo He, Nanjing (CN); Hong Li, Nanjing (CN); Cheng-Hsien Wu, Piscataway, NJ (US); Chuan Sun, Piscataway, NJ (US)

(73) Assignees: Nanjing GenScript Biotech Co., Ltd., Nanjing (CN); GenScript USA Inc., Redmond, WA (US)

(21) Appl. No.: 18/042,066

(22) PCT Filed: Aug. 17, 2021
(86) PCT No.: PCT/CN2021/112896

§ 371 Date: Feb. 17, 2023

(30) Foreign Application Priority Data
Aug. 17, 2020 (WO) PCT/CN2020/109583

(51) Int. Cl. C12N 15/10; C12M 1/00

(52) U.S. Cl. CPC C12N 15/1006; C12M 47/06

(57) ABSTRACT

Provided is an automated system for preparing purified plasmid DNA in commercial scale.

### FIELD OF INVENTION

This invention is related to an automated system for preparing purified plasmid DNA in commercial scale.

### BACKGROUND OF THE INVENTION

The current tremendous increase in demand for plasmids is linked to the growing cell and gene therapy sectors. Gene therapy generally has a viral vector as one of its main components, and viral vector production relies on plasmids. The resultant backlog in production is likely to become more severe as demand increases for starting materials, the intermediates of production, and the final means of transduction or transfection. It is suggested that the major risk faced today by plasmid manufacturers is an inability to serve the industry properly and at the right time with specific plasmids. This could result in a slowing of progress in R&D pipelines worldwide, and negatively affect the expectations both of the market and of the patients who are waiting for new products and solutions for their specific needs.

To produce plasmids in the necessary quantities in a timely fashion, automation and integration of the required manufacturing equipment is a critical requirement. In addition, to produce high quality plasmids, equipment and operations must be designed to prevent cross-contamination. For this, it is essential that the system be controlled and operated remotely to reduce operator intervention. This system disclosed herein is designed for automated continuous production of scalable quantities of plasmids and is controlled and operated by computer. This design will address the issues of time, quantity, and risk of cross-contamination.

### BRIEF SUMMARY OF THE INVENTION

Provided herein is an automated plasmid preparation module, comprising multiple operation units and at least one multi-axis robot, wherein, the multiple operation units comprise at least A) a culture and lysis unit for culturing and lysing bacteria transformed with plasmid, B) a purification unit for obtaining purified plasmid DNA, and C) an optional quality control unit; and the multi-axis robot is programmed to control and operate the multiple operation units.

In one embodiment of the automated plasmid preparation module, the module further comprising a track, on which the multi-axis robot moves between the multiple operation units.

In a further embodiment of the automated plasmid preparation module, the multi-axis robot comprises a robot arm, a holding means for tube and bottle, a capping/decapping means, a shaking mechanism, one or more liquid dispensing units, and sensor(s) for receiving signals from the multiple operation units.

In a yet further embodiment of the automated plasmid preparation module, the A) culture and lysing unit comprises a sequence of incubators with shaking rack(s), one or more centrifuges, and at least one sterilizer, and the B) purification unit comprises one or more vacuum manifolds, one or mom centrifuges, one or more column trays each holding a set number of DNA columns, wherein each DNA column has an open upper end, an open lower end, a solid phase capable of plasmid DNA capture, and a filter above the solid phase.

Further provided herein is an automated plasmid preparation system comprising one or more of the automated integrated plasmid preparation module described above.

Yet further provided herein is an automated plasmid preparation process utilizing the automated plasmid preparation system described above, wherein the multi-axis robots are programme to operate each of the modules simultaneously.

Yet further provided herein is an automated plasmid preparation process utilizing the automated plasmid preparation system described above, wherein, the multi-axis robot is programmed to,
A) culture and lyse plasmid DNAs, utilizing the culture and lysing unit; and
B) purify the plasmid DNAs, utilizing the purification unit.

In one embodiment of the automated plasmid preparation process, wherein, in the A) culturing and lysing step, each multi-axis robot is programmed to,
a) receiving a sequence of incubators each holding a set number of empty culture bottles;
b) sequentially for each incubator at a time, filling each of the culture bottles with growth media and inoculating bacteria samples transformed with plasmid;
c) upon completion of incubation of one of the sequence of incubators from step b), retrieving one or more of the culture bottles and loading them into the centrifuge;
d) upon completion of centrifugation, retrieving one culture bottle from the centrifuge and decanting the supernatant;
c) sequentially adding resuspension reagent, lysis reagent, and neutralization reagent into the culture bottle from step d), each addition followed by gentle shaking;
f) placing the culture bottle from step e) in the centrifuge;
g) repeating steps d)-f) until the centrifuge is full.

In a further embodiment of the automated plasmid preparation process described above, in the B) purifying step, each multi-axis robot is programmed to,
h) retrieving equilibrated DNA columns and after step g) and upon completion of centrifugation, sequentially retrieving each of the culture bottles from the centrifuge and moving the supernatant on top of the filter into each of the equilibrated DNA columns;
i) placing the DNA columns on top of vacuum manifold which is capable of holding a set of DNA columns;
j) applying pressure on the DNA columns and discarding the elute;
k) adding wash buffer into the DNA columns, applying pressure on the DNA columns, and discarding the filter and elute;
l) optionally repeating step k) 1-3 times;
m) for each one of the DNA binding columns, placing one collection bottle underneath thereof;
n) adding elution buffer in each of the DNA columns from step m) and applying pressure on the DNA columns to elute aqueous sample in the collection tubes;
o) adding isopropyl alcohol in each of the collection tubes from step n) followed by vortexing;
p) sequentially placing each of the collection tubes from step o) in the centrifuge until centrifuge is full;
q) upon completion of centrifugation, sequentially retrieving each of the collection tubes from the centrifuge and decanting supernatant;
r) sequentially adding water into each of the collection tubes from q) to resuspend sample and removing each of the sample suspensions to each of a series of sample vials.

In a yet further embodiment of the automated plasmid preparation process described above, after step b), each of the sequence of incubators is programmed to sense the completion of inoculation and start incubation or the multi-axis robot is programmed to signal each of the sequence of incubators to start incubation.

In a yet further embodiment of the automated plasmid preparation process described above, after step c) and after step g), the centrifuge is programmed to sense the completion of loading to start centrifuge or the multi-axis robot is programmed to signal the centrifuge to start centrifugation.

In a yet further embodiment of the automated plasmid preparation process described above, after step i), if the vacuum manifold is not full,
i) if the centrifuge from step d) is not empty, repeating steps d)-i);
ii) if the centrifuge from step d) is empty and the one of the sequence of incubators from step c) is not empty, repeating steps c)-i);
iii) if the centrifuge from step d) is empty, the one of the sequence of incubators from step c) is empty, and the next incubator in sequence has sent completion signal, repeating steps c)-i); and
iv) if the centrifuge from step d) is empty, the one of the sequence of incubators from step c) is empty, and the next incubator in sequence has not sent completion signal, wait until the next incubator in sequence has sent completion signal to repeat steps c)-i).

In a yet further embodiment of the automated plasmid preparation process described above, after step r), if there are remaining culture bottles from step c), repeating steps c)-r); and if there are no remaining culture bottles from step c), repeating steps a)-r) or coding process.

In a yet further embodiment of the automated plasmid preparation process described above, after step h), the multi-axis robot is further programmed to place the culture bottles to recycling station for cleaning and reuse.

In a yet further embodiment of the automated plasmid preparation process described above, after step k) or l), the multi-axis robot is further programmed to dry the DNA column by blotting and/or vacuum.

In a yet further embodiment of the automated plasmid preparation process described above, after step n), the multi-axis robot is further programmed to discard the used DNA columns and remove the column tray.

In a yet further embodiment of the automated plasmid preparation process described above, after step q), the multi-axis robot is further programmed to wash the pellets remaining in the collection tube with 70% ethanol 1-3 times.

In a yet further embodiment of the automated plasmid preparation process described above, after step r), the multi-axis robot is further programmed to remove a portion of the sample in each sample vial to microplates for quality control.

### BRIEF DESCRIPTION OF THE DRAWING

FIG. 1 is a workflow chart of an exemplary automated plasmid preparation module disclosed herein.

### DETAILED DESCRIPTION OF THE INVENTION

Disclosed herein is an automated continuous system for preparing scalable quantities of plasmid DNA in a scheduled fashion. The automated system includes one or more automated plasmid preparation modules, where each module comprises one or more of the following: a track, a multi-axis robot, an incubator/shaker, an indexing centrifuge, a sterilizer, and a manifold with decapper, capper, liquid dispenser, and pick-up tool for the robot arms. Associated apparatus may also include one or more of the following: rack, microplate reader, autoclave, manifold jig, solution container, cart, and waste chute. In each system, a plurality of automated units may be built as needed. This system rapidly delivers large-scale quantities of purified plasmids for applications known to persons skilled in the art.

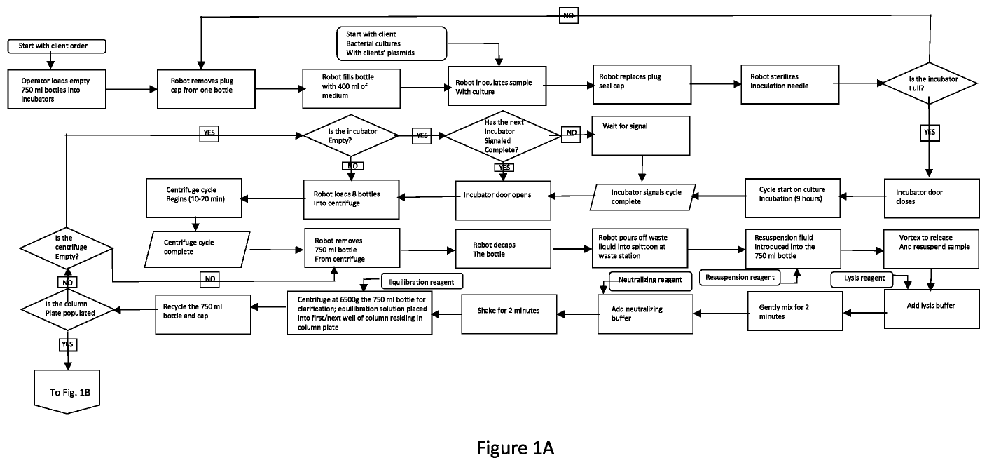

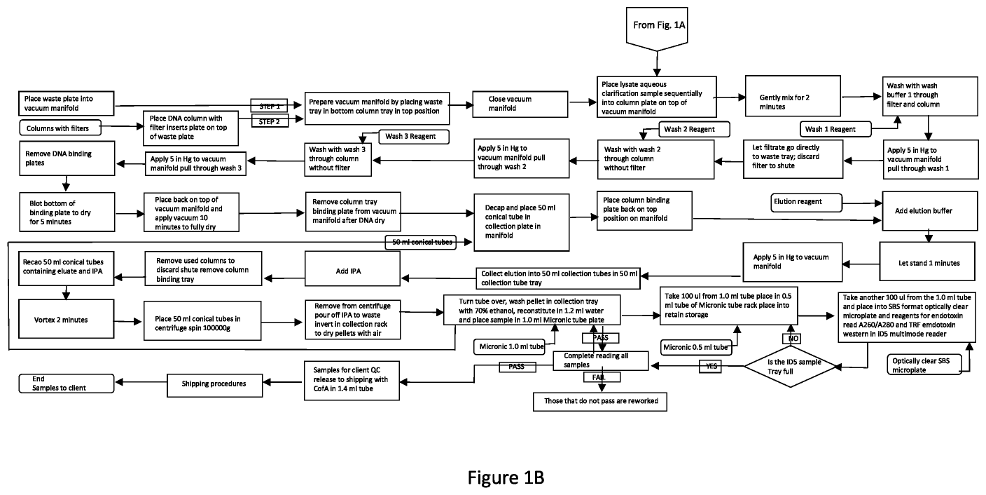

FIG. 1 is an outline of one exemplary embodiment of the system, which will be described in detail below.

In general, the automated system disclosed herein may be formed of multiple modules and each module may comprise the following components: track, multi-axis robot, incubator/shaker, centrifuge, sterilizer, and manifold with decapper, capper, liquid dispenser, pick-up tools for the robot arms, etc.

Examples of the components that can be used in the 6-Axis Robotic Arm System (2 systems) are provided below (Some integration may be using out of box items with Macherey-Nagel kits).
LR Mate 200iD/7LC clean room/food series/pharmaceutical series 6-axis robot with R-30iB plus controller for FANUC robots; one for each system;
Innova 40R incubated shaker;
Hettich Rotanta 460R indexing centrifuge, 4 per system;
Farason custom manifolds; 4 per system;
Custom gripper for 500-mL Corning bottles also with liquid dispensing unit capper/decapper for 500/50-mL conical tubes, custom waste chute; one per system.
One SpectraMax iD5 multi-mode microplate reader;
Polyetheretherketone (PEEK) Manifold jigs 12 per system;
Stainless steel reagent cart;
Stainless steel waste cart;
AEC Floor to ceiling HEPA Hood.
Autoclave;
GETINGE 433HC Vacuum/Gravity Steam Sterilizer;
16 incubators per system, each incubator holds 16 bottles (each bottle one maxiprep) in 2 racks (in two decks) processed in scheduled fashion, bottom rack first (4 modules; 256 maxipreps) then top rack (4 modules; another 256); for 512 maxipreps.
To produce at least 1000 plasmid samples would require 2 systems per 24 h.
Macherey-Nagel kits (25 maxis per kit), 40 kits;
Corning polypropylene 500-mL conical-bottom sterile disposable tubes (holding jig included);
Gas permeable caps for Corning bottles, 1024 caps.
Corning 50-mL conical receiving tubes for incubator, 1024 tubes.

The following are components needed for robot to be able to conduct the incubating, harvesting and extracting steps continuously in a reliable and reproducible fashion.

Incubation requires rapid agitation of reactor bottles with vented porous plug caps on shaker tables with bottles canted to side to achieve an OD of 3 in less than 10 hours. This incubation is accessed by the robotic arm after completion, and, in scheduled staggered fashion, is brought to several indexing centrifuges that allow for rapid pelleting of bacteria with plasmid at optimal density of 3 to 6 OD and resuspension with contamination-free dispense-only addition of solutions. The plasmid preparation process proceeds by incubation of resuspension solution and requires metered vortexing so that plasmid samples are not damaged. Variable-speed vortex measures are ideally suited for robotic arm finesse vortexing using optics to register when pellets have been resuspended. Optimal timing with the robotic arm also allows rapid contamination-free dispensing of lysis solution. Gentle mixing with roller bottle completes release of plasmid and disruption of cells. Centrifugation is performed in a second bank of indexing centrifuges to prevent bottlenecking. Pouring into the column manifold is performed with the filter in place to allow for scheduled pouring into the column. Rapid dispense of wash 1 from the robotic arm allows accurate splash-free dispense-only addition. Removal of filters requires accurate gripper tools that are cleaned after each filter removal. The clean tool is brought back into position and the platinum gripper tools are cleaned with bleach/ethanol/water and heat. Washes 1 and 2 are performed on the silica column with bound plasmid DNA using dispense-only addition, and the elution buffer is added with precision to properly extract all plasmid with positive oil-free sterile air push from column top. The resulting plasmid is processed for low-volume removal of endotoxin by repeat ethanol precipitations.

The robotic manipulator will deploy intelligent machine vision to guide the robot to the pickup locations for: the incubator trays full of cultures to process, the individual bioreactor bottles after they are spun in the centrifuges that may stop in any location, the pickup location for the pour-off rack, and the location of the filter inserts of the columns that will be discarded. Sophisticated End-of-Arm Tools featuring quick-change capability will be utilized for the varied tasks to be performed. Aside from the primary robotic manipulator, multi-axis linear servo systems will control the dispensing nozzles, capper/uncapper, and rack motion to allow for asynchronous indexing of the gravity-driven process. Also, specialized platinum inoculation and sampling tools will be paired with an induction sterilization unit for inoculating the initial growth flasks, as well as depositing post-process samples for QC testing.

Operation of the system is designed to eliminate bottlenecks and uses a conventional system of vented bottles with high asymmetric shaking using large throw shaker incubators in scheduled mode to create optimal high agitation and aeration for large numbers of reactor bottles. The rapid incubation coupled to optimal rapid access to indexing centrifuges allows for rapid pelleting of staggered sets. These sets allow for production of large number of plasmids in 24 hours. The number of incubators and centrifuges is optimized by using 4 shifts, optionally recycling the bottles after cleaning. The barcode tracking system assigns a new bar code for each plasmid preparation. The manifolds are designed so as the 4 shifts of lysed neutralized pellets are processed the reagents are added as dispense-only, eliminating cross-contamination and processing 250 columns at a time in four jigs. These jigs can be set while the robot is continually running thus increasing speed and number of samples. Reloading reagents and plasticware will be done upon remote sensing and text informing when volumes are low. A housing can be placed over the robot for increasing air quality in production.

This system is designed to operate with limited operator intervention. Further development phases of this system will reduce resource and subsequent environmental requirements even further by operating in a fully isolated environment. Engineers will establish the most optimum rates of operation for the included motion systems for system balance and component lifespan. Based upon process inputs mentioned earlier (type of culture media, antibiotics, bacterial host strain, plasmid type, size, and copy number) the growth and purification critical process parameters, CPPs, will be configured into programs that can be selected by the operator through the User Interface, or automatically depending upon system configuration.

The automated integrated system disclosed herein may need limited operator invention, such as the following;
Input new bottles, caps, filters, and columns, etc. as needed;
Change media supply bottles;
Load/unload wash rack for reusables;
Remove finished product from the end of line;
Monitor system parameters and make any program selections based on production demand;
Set up programs for new product configurations;
Dispose of waste, etc.

In one embodiment, the system starts by the preparation of culture samples. For example, a plasmid sample is obtained and placed onto the deck of the storage and retrieval/pre-culture liquid handler robot wherein 2 μL thereof is automatically transformed into E. coli in a deepwell plate. The E. coli then receives a heat shock and 2 mL SOC medium followed by incubation at 37° C. for 2 hours. Thereafter, the transformed E. coli is autospread onto a growth medium plate (e.g., LB antibiotic Omnitray plate) and incubated at 37° C. for 24 hours. 2 mL of the cultured colony is picked into a 96-well deepwell plate with each well filled with growth medium (e.g., LB broth) and followed by incubation at 37° C. for 5 hours. Then, using sterile technique, an operator moves the deepwell plate with about 1 mL of the culture to one module of the plasmid preparation automated integrated production line to inoculate 400 mL of growth medium (optionally with antibiotic) in 500 mL conical flasks on the plasmid prep multi-axis pharmaceutical robot. The system may perform 32 maxi-preps per module and incorporates 4 modules for 128 plasmid preps. The robot receives the plate having verified that the transformation grew, was not contaminated, and has been checked using PCR for quick verification of the presence of the correct plasmid sequence. The required number of clean 500-mL Corning disposable plastic conical bottom flasks are loaded onto the industrial plasmid maxi preparation production system at prompt. The following equipment and solutions are also loaded: plasmid prep disposable plastic ware for the filter and the silica column from the Macherey-Nagel kit; clean growth medium with antibiotic to the medium dispenser unit; Macherey-Nagel bulk solutions for resuspension with RNAse A added; lysis, equilibration, and neutralization buffers to their clean dispenser units; wash solutions, 50-mL sample Falcon conical tubes, isopropyl alcohol and the 70% ethanol to their Peltier chilled dispense stations, and Tween detergent for endotoxin removal steps. The deepwell plate with the cultures for the number of sample preps to be performed are then loaded by entering the plate barcode into the production system with a Zebra barcode unit.

The robot is programmed to initiate the run by filling the appropriate number of Corning 0.22-micron filter cap 500-mL conical-bottom tubes from the first of the four 32-process modules. The robot sequentially decaps each 500-mL conical bottle employing a capper/decapper unit using the 6-axis arm with the Corning bottle gripper tool. The decapped bottle is filled at the bell-covered sterile filling station with 400 mL of growth medium with antibiotic. A selector valve on the clean medium filler unit can choose among the various media required. The Corning 500-mL bottle is clean filled with dispense-only nozzles then set on the plasmid processing area and inoculated with the picking tool on the 6-axis arm. A clean tip is used to pick and dispense-only the inoculum from the 96-well deepwell plate into the 400-mL medium in the conical-bottom 500-mL Corning bottle. This is repeated until all 32 bottles in the first module have been inoculated and placed into two I24 Innova shaker incubators, opened by the robot, that can each hold sixteen 500-mL conical-bottom flasks in conical adaptors on the Innova shaker deck. They are set to shake 100 rpm at 37° C. on the Innova 124 shakers, and the housing is closed. Shaking is continued for 9 hours with a 1-mL culture inoculation. The next three modules will be started in series in a scheduled fashion and will also be shaken for 9 hours.

After the incubations are complete, the bottles are removed by the robot into 4 positions of each of 4 indexing centrifuges. These centrifuges will pellet the bacteria in each of the first 16 cultures into the conical bottoms for concentration and better processing. The bottles will be spun in the indexing centrifuge at 6,000 rpm for 12 minutes at 4° C. The cultures will then be decanted with the robotic arm into the quick waste disposal unit to a waste carboy for removal after the run. The evacuation of the medium will be accomplished with a 360-degree dump executed by the gripper tool into a positive-air-in housing to prevent splatter using a compressed air line with a dryer, a 0.22 micron filter to remove microbes, and a 0.01 micron filter for oil removal. Each bottle of the first Innova shaker will be processed. Each of the 4 modules has two Innova 124 shakers with 16 conical-bottom jars. After the first 16 are decanted, the second set of 16 from the first module are spun and decanted similarly. The pellets will be resuspended by the robotic arm by putting the bottle under the dispenser on the robotic arm and adding 12 mL resuspension buffer into the conical bottle. The pellets will be resuspended in the Innova shakers at 300 rpm with all 32 shaken rapidly after this dispense-only step to pull up the pellets.

The pellet will be lysed by dispensing 12 mL of lysis buffer also plumbed to the dispense tool on the robot arm. All 32 conical-bottles will then be shaken with finesse so as not to damage supercoiled plasmid. The shaking will last for 30 minutes using a combination of shaking speeds from 50 to 400 to 500 rpm until lysis is complete, then equilibration solution will be added to the tube by the robotic arm with a plumbed line for dispense-only of the 25 mL of equilibration buffer to all 32 bottles. Shaking will proceed for 15 minutes until mixed. Then 12 mL of neutralization solution will be added from the dispense arm for all 32 bottles in the first module. This lysed material after neutralization will be centrifuged in all four indexing centrifuges for the first 16 samples and then the second 16 will be performed. While centrifugation occurs, prewetting of the clean NucleoBond Xtra Column filters with 2 mL of neutralization buffer from the robotic arm will be performed. Each of the lysates will be clarified by pouring into the autoclaved clarification NucleoBond Xtra Column filter jig with prewetted filters in place. After the material from all 32 units of the first module has been poured into the filters, a sterile press of compressed filtered clean air will push the material gently through the silica columns.

After clarified material is pushed (not aspirated to avoid contamination) through the NucleoBond Xtra Column filter jig with prewetted filters with positive pressure manifold press, the material will pass through narrow nozzles into the center of the NucleoBond Xtra Column and a second pressure manifold slides over the top of the columns and presses the material through the columns where the DNA binds. After the DNA is bound, pressure will be maintained until the column is dry. After air drying using gentle compressed clean sterile air, the manifold will be slipped back, and the robot arm will be brought in to dispense the first wash solution of 15 mL to all 32 samples in the first module. The second manifold will be restored and will press the wash solution through the column. The second manifold will be moved back once more automatically and 25 mL of the second wash solution will be added to all 32 columns, and the second manifold press will be put in place again and will press the wash through.

Samples are air dried for a period of 5 minutes. Then the manifold will be pulled back and 15 mL of elution buffer will be added to each column. The second manifold will be added to the columns containing elution buffer, and the elution will be allowed to proceed slowly with clean sterile compressed air. The resulting eluate will pass into the catch jig and the first and second pressure manifolds will be automatically pulled back to reveal the DNA plasmid samples in their respective 50-mL conical tubes. Cold 100% isopropyl alcohol will be added to each tube and the tubes will be capped at the capper station and placed into the Hettich centrifuge set up with 50-mL conical adaptor and spun down. The isopropyl alcohol will be decanted into the waste evacuation system for each of the 32 samples in the module and the samples will be rinsed with 70% ethanol. The 70% ethanol will be decanted to the waste evacuation system. The tubes will be allowed to air dry for 30 minutes then will be reconstituted in 5 mL of Tween solution to remove endotoxin. The solution will be precipitated again with 45 mL of cold 100% isopropyl alcohol, capped, shaken, and then centrifuged at 10,000 rpm in the Hettich centrifuge. All tubes will be washed with 10 mL 70% ethanol, centrifuged at 10,000 rpm, and decanted to the waste evacuation unit. The final pellet will be reconstituted in 1.2 mL 1×TE buffer in 1.4 mL vials for retain storage, QC evaluation, and shipment to customers. Specifically, 100 μl of the final plasmid sample from each vial is transferred into 0.5 mL micro tubes for retain storage, and another 100 μl is transferred into optical clear microplate and fed into ID5 readers for QC evaluation, e.g., concentration and/or contamination of protein or endotoxin. For samples that failed the QC evaluation, the vials that contain the failed samples will be reworked. Moreover, throughout the process, intermediate QC evaluation can be performed during any step of the process, for example after the lysing step and before the purification step.

When purifying plasmid DNA from E. coli, the first step is cell growth. A person of skill in the art can select the appropriate medium and growth conditions depending on the cell type, number of samples, desired yield, etc. Culture media can be chosen based on the bacterial strain. A chemically defined (synthetic) medium is one in which the exact chemical composition is known. A complex (undefined) medium is one in which the exact chemical constitution of the medium is not known. Defined media are usually composed of pure biochemicals off the shelf; complex media usually contain complex materials of biological origin such as peptone, tryptone, blood, milk, yeast extract or beef extract, the exact chemical composition of which is undetermined. Complex media usually provide the full range of growth factors that may be required by an organism so they may be more handily used to cultivate unknown bacteria or bacteria whose nutritional requirements are complex (i.e., organisms that require a lot of growth factors, known or unknown).

During the culture step, any suitable growth medium may be used. In general, a complex rich medium may be used for cell growth. Complex media include LB, Terrific Broth, SOC, SOB, YT, 2×YT, Agencourt Ale (Beckman Coulter), Plasmid Plus (Thompson Instrument Company) and others. As disclosed herein, a rich medium will be defined as belonging to the group consisting of Terrific Broth (TB), SOB, SOC, YT, 2×YT, NZCYM, Agencourt Ale, CIRCLEGROW® (MP BIOMEDICALS), PDM (0.79% Tryptone, 0.44% Yeast extract, 1.0% Glucose, 1.28% Disodium phosphate 7H₂O, 0.3% Monopotassium phosphate, 0.024% Magnesium sulfate, 0.05% Ammonium chloride), EnPresso and mixtures thereof. For plasmid purification, the growth medium additionally contains the appropriate antibiotic for maintaining the plasmid.

A person of skill in the art can also select the appropriate growth conditions for a given bacterial strain.

The resuspension buffer used herein may be comprised of IM Tris-HCl pH8.0, IM EDTA, and 4 mg/mL RNase A.

The term “lysis” or “lysed” is a process by which cells are treated to break the cell wall or membrane and release the nucleic acids.

Lysis can be accomplished by a number of means including physical or chemical action. Non-limiting examples of lysis methods include mechanical, such as ultrasonic waves, mortar and pestle, osmotic shock, chemical e.g. by means of detergents and/or chaotropic agents and/or organic solvents (e.g. phenol, chloroform, ether), heat and alkali.

In accordance with the present disclosure, lysis via chemical means is used, which involves the addition of a lysis solution to the resuspended cells. The lysis buffer used herein may be comprised of 6M NaOH and 10% SDS.

The lysis procedure is followed by the addition of a neutralization buffer (also known as a precipitation solution). The neutralization buffer may contain an acid. For example, the neutralization buffer used herein may be composed of 10 M guanidine hydrochloride and 5M potassium acetate pH 4.5.

The neutralization buffer may also contain a chaotropic agent and/or other components. A chaotropic agent is a molecule in water solution that can disrupt the hydrogen bonding network between water molecules (i.e. it exerts chaotropic activity). This has an effect on the stability of the native state of other molecules in the solution, mainly macromolecules (proteins, nucleic acids) by weakening the hydrophobic effect. Chaotropes can be complexed with an alcohol or a salt. Examples of chaotropic reagents include sodium iodide, sodium perchlorate, guanidine thiocyanate (GuSCN), urea, guanidine hydrochloride (GuHCI), potassium iodide, sodium perchlorate, potassium chloride, lithium acetate, lithium chloride, magnesium chloride, sodium chloride, butanol, ethanol, phenol, propanol, sodium dodecyl sulfate, thiourea, urea or mixtures of such substances. These chaotropes may have other properties. For example, butanol and ethanol are solvents. Sodium dodecyl sulfate is a surfactant.

The DNA columns used herein are comprised of a column body having an open upper end, an open lower end, and an open charnel between the upper and lower ends of the column body. Within the open channel, towards the bottom end, it is filled with a solid phase (or media) capable of binding DNAs. Optionally, a filter is placed with the channel, towards the upper end, to capture cell debris.

The media or solid phase used in the column can be a form of water-insoluble particle (e.g., a porous or non-porous bead, fiber or other particle) that has an affinity for the nucleic acid of interest. Silica (SiO₂) is one of the most widely used chromatography sorbents and is found in nature most often as quartz. Silica beads are suitable for the columns of the invention. Silica grades such as Davisil 923 and 635 work well. Other suitable materials include celite, diatomaceous earth, silica gel, silica gel, (Davisil, Impaq, Biotage), metal oxides and mixed metal oxides, glass, alumina, zeolites, titanium dioxide, zirconium dioxide. Ion exchangers made of inorganic or polymeric substrates also work quite well.

The beads or particles used in the column have a shape or pore structure that provides a large surface area or exposed surface. In some embodiments, the capture material has a surface area of greater than 0.5 m², 1 m², 1.5 m², 2 m², 3 m², 5 m², 6 m², m², 10 m², 20 m², or 30 m² per gram of material.

The bed volume of the medium used in the columns of the invention depends on the scale of the plasmid purification. In certain embodiments, the bed volume can be in the range of 0.2 mL to 20 mL. In terms of percentage, the bed volume can be in the range of 5% to 50% of the column volume. In certain embodiments, the bed volume can be in the range of 7%-40% of the column volume or in the range of 10%-30% of the column volume.

The space between resin particles can also be important. This space increases with looser packing of the column. In certain embodiments, the column beds ae not tightly packed.

In other embodiments, the plasmid DNA can bind to a membrane such as a silica membrane in the column. DNA binds to silica in the form of particles, beads, gels or fibers. Membranes may consist of fibers or a mixture of particles and fibers. Silica may be bound with a binding agent to form a membrane. The materials may be packed as particles into columns or formed as membranes and then placed into columns. Membranes are porous so that liquids can pass through the column.

Ion exchange resins can also be used for plasmid purification. Although it is not always the case, some anion exchange resins are used in gravity-flow, liquid chromatographic columns containing porous silica beads or other types of polymer or inorganic base media modified with diethylaminoethanol or another strong or weak base anion exchanger. Any anion exchange group may be used. Anion exchange bed volumes can be larger than the bed volumes used in silica-based columns.

One or more frits are used to contain the bed of medium in a column. Frits can take a variety of forms and can be constructed from a variety of materials. The frits of the invention are porous, since it is necessary for fluid to be able to pass through the frit. The frit should have sufficient structural strength and integrity to contain the extraction media in the column. It is desirable that the frit have little or no affinity for chemicals with which it will come into contact during the extraction process, particularly the analyte of interest. Frits of various pores sizes and pore densities may be used provided the free flow of liquid and particulates is possible. Frits of pore size large enough to prevent plugging from cell debris are of particular interest. Some frits of the invention have a large pore size frit.

In one embodiment, a single frit (e.g., a lower, or bottom, frit) extends across the open channel of the column body. Often, the bottom frit is attached at or near the open lower end of the column, e.g., extending across the open lower end. This configuration is not required, i.e., in some embodiments, the bottom frit is located at some distance up the column body from the open lower end. Normally, a bed of medium is positioned inside the open channel in contact with the bottom frit.

In certain embodiments, a top frit may be employed. For example, in some embodiments, a second frit extends across the open channel between the bottom frit and the open upper end of the column body. In this embodiment, the top frit, bottom frit and column body (i.e., the inner surface of the channel) define a media chamber wherein a bed of medium is positioned. The frits should be securely attached to the column body and extend across the column body to completely occlude the channel, thereby substantially confining the bed of medium inside the media chamber.

In some embodiments, the top frit can be just above the bed of medium or in contact with the bed of medium. In other embodiments, the top frit is positioned well above the medium, e.g., 25 mm or more above the medium in a 200 μL pipette tip column or 50 or more mm above the bed in a 1.2-mL pipette-tip column. The position of the top frit can be proximal to the open upper end of the pipette tip column. That is, the top frit can be closer to the open upper end of the column than to the bed medium. In these embodiments, the bed is not packed, and the medium can occupy well under 50% of the volume of the extraction media chamber and the top frit can be significantly thicker than the bottom frit. In some embodiments, liquids may not flow through the top frit.

The position of the top frit over the bed may just touch the top of the resin bed or be positioned substantially above the resin bed. When the frit is above the resin bed, the resin bed may move or expand with aspiration of liquids including the sample containing the particulates. The bed may move down against the bottom frit with expulsion of the liquid.

The performance of the column is typically enhanced by the use of frits having pore or mesh openings sufficiently large to allow cell debris or other particulates to flow through the frit without clogging or plugging under low pressures applied by a pipette or liquid handler. Of course, the pore or mesh openings should not be so large that they are unable to adequately contain the extraction media in the chamber. Frits used on columns of the invention can have pore openings or mesh openings of a size in the range of about 5-500 μm, more preferably 10-200 μm, and still more preferably 100-150 μm, e.g., about 120 μm.

In some cases, it is necessary to consider the relationship between the frit pore size and the particle diameter. Specifically, it is possible to increase the frit pore size when the particle diameter is increased. For example, a frit pore size of 100 μm was used successfully with a range of different resins.

Some embodiments of the columns of the invention employ a thin frit, preferably less than 2000 μm in thickness (e.g., in the range of 20-2000 μm, 40-350 μm, or 50-350 μm), more preferably less than 200 μm in thickness (e.g., in the range of 20-200 μm, 40-200 μm, or 50-200 μm), more preferably less than 100 μm in thickness (e.g., in the range of 20-100 μm, 40-100 μm, or 50-100 μm). However, thicker frits, up to several mm, 5 and even 10 mm, thick may be used if the pore size of the frit can be increased dramatically.

Some embodiments of the invention employ a membrane screen as the frit. The use of membrane screens as described herein typically provide this low resistance to flow and hence better flow rates, reduced backpressure and minimal distortion of the medium. The membrane can be a woven or non-woven mesh of fibers that may be a mesh weave, a randomly orientated mat of fibers i.e. a “polymer paper,” a spun bonded mesh, an etched or “pore drilled” paper or membrane such as nuclear track etched membrane or an electrolytic mesh (see, e.g., U.S. Pat. No. 5,556,598). The membrane may be, e.g., polymer, glass, or metal provided the membrane is low dead volume, allows movement of the sample and various processing liquids through the column bed, may be attached to the column body, is strong enough to withstand the bed packing process, is strong enough to hold the column bed of beads, and does not interfere with the extraction process i.e. does not adsorb or denature the sample molecules.

The frit can be attached to the column body by any means that results in a stable attachment. For example, the screen can be bonded to the column body through welding or gluing. The column body can be welded to the frit by melting the body into the frit, or melting the frit into the body, or both. Alternatively, a frit can be attached by a friction fit or by means of an annular pip, as described in U.S. Pat. No. 5,833,927.

The frits of the invention can be made from any material that has the required physical properties described herein. Examples of suitable materials include polymer, sintered polymer, fiber, nylon, polyester, polyamide, polycarbonate, cellulose, polyethylene, nitrocellulose, cellulose acetate, polyvinylidine difluoride, polytetrafluoroethylene (PTFE), polypropylene, polysulfone, PEEK, PVC, vinyl polymer, metal (e.g., steel), ceramic and glass.

In certain embodiments of the invention, a wad of fibrous material is included in the column, which extends across the open channel below the open upper end of the column body, wherein the wad of fibrous material and open channel define a media chamber, wherein the medium is positioned within the media chamber. This wad of fiber can be a porous material of glass, polymer, metal, or other material having large pores. In some embodiments, the wad of fibrous material is used in lieu of an upper frit.

The 6-axis arm on a track platform can provide all operations for 1024 low-cost plasmid preps very quickly in a scheduled fashion on two redundant systems with few disposable input costs for the 1 mg overnight plasmid prep market. The units can be loaded and operate unattended overnight performing the Macherey-Nagel format maxi prep scale plasmid prep without any cross contamination for very little cost. Scaling two 4-module 6-axis robotic arms can give rise to 1024×2 mg maxi prep kits with the quality of the debris removal and the removal of endotoxin improved at extra cost and time. These units are robust and can be built as needed by industrial automation groups. It is also understood that the modules and systems disclosed here may be further modified to prepare mega prep or giga prep scale plasmid prep.

### Embodiments

The invention provides also the following non-limiting embodiments.

Embodiment 1 is an automated plasmid preparation module, comprising multiple operation units and at least one multi-axis robot, wherein,
the multiple operation units comprise at least A) a culture and lysis unit for culturing and lysing bacteria transformed with plasmid, B) a purification unit for obtaining purified plasmid DNA, and C) an optional quality control unit; and
the multi-axis robot is programmed to control and operate the multiple operation units.

Embodiment 2 is the automated plasmid preparation module of embodiment 1, which further comprises a track, on which the multi-axis robot moves between the multiple operation units.

Embodiment 3 is the automated plasmid preparation module of embodiment 1 or 2, wherein, the multi-axis robot comprises a robot arm, a holding means for tube and bottle, a capping/decapping means, a shaking mechanism, one or more liquid dispensing units, and sensor(s) for receiving signals from the multiple operation units.

Embodiment 4 is the automated plasmid preparation module of any one of embodiments 1-3, wherein, the A) culture and lysing unit comprises a sequence of incubators with shaking rack(s), one or more centrifuges, and at least one sterilizer, and the B) purification unit comprises one or more vacuum manifolds, one or more centrifuges, one or more column trays each holding a set number of DNA columns, wherein each of the DNA column has an open upper end, an open lower end, a solid phase capable of plasmid DNA capture, and a filter above the solid phase.

Embodiment 5 is the automated plasmid preparation module of embodiment 4, which comprises one incubator capable of holding 32 culture bottles.

Embodiment 6 is the automated plasmid preparation module of embodiment 5, wherein each of the culture bottles is a 500-mL culture bottles, and wherein, the module can produce 32 maxi preps of plasmid samples.

Embodiment 7 is an automated plasmid preparation system comprising one or more of the automated plasmid preparation module of any one of embodiments 1-6.

Embodiment 8 is an automated plasmid preparation system of embodiment 7, which is comprised of 4 automated plasmid preparation modules of any one of embodiments 1-6.

Embodiment 9 is an automated plasmid preparation process utilizing the automated plasmid preparation system of embodiment 7 or 8, wherein the multi-axis robots are programmed to operate each of the modules simultaneously.

Embodiment 10 is an automated plasmid preparation process utilizing the automated plasmid preparation system of embodiment 7 or 8, wherein, the multi-axis robot is programmed to,
A) culture and lyse plasmid DNAs, utilizing the culture and lysing unit; and
B) purify the plasmid DNAs, utilizing the purification unit.

Embodiment 11 is an automated plasmid preparation process of embodiment 10, wherein, in the A) culturing and lysing step, each multi-axis robot is programmed to,
a) receiving a sequence of incubators each holding a set number of empty culture bottles;
b) sequentially for each incubator at a time, filling each of the culture bottles with growth media and inoculating bacteria samples transformed with plasmid;
c) upon completion of incubation of one of the sequence of incubators from step b), retrieving one or more of the culture bottles and loading them into the centrifuge;
d) upon completion of centrifugation, retrieving one culture bottle from the centrifuge and decanting the supernatant;
e) sequentially adding resuspension reagent, lysis reagent, and neutralization reagent into the culture bottle from step d), each addition followed by gentle shaking;
f) placing the culture bottle from step e) in the centrifuge;
g) repeating steps d)-f) until the centrifuge is full.

Embodiment 12 is the automated plasmid preparation process of embodiment 10 or 11, wherein, in the B) purifying step, each multi-axis robot is programmed to,
h) retrieving equilibrated DNA columns and after step g) and upon completion of centrifugation, sequentially retrieving each of the culture bottles from the centrifuge and moving the supernatant on top of the filter into each of the equilibrated DNA columns;
i) placing the DNA columns on top of vacuum manifold which is capable to hold a set of DNA columns;
j) applying pressure on the DNA columns and discarding the elute;
k) adding wash buffer into the DNA columns, applying pressure on the DNA columns, and discarding the filter and elute;
l) optionally repeating step k) 1-3 times;
m) for each one of the DNA binding columns, placing one collection bottle underneath thereof;
n) adding elution buffer in each of the DNA columns from step m) and applying pressure on the DNA columns to elute aqueous sample in the collection tubes;
o) adding isopropyl alcohol in each of the collection tubes from step n) followed by vortexing;
p) sequentially placing each of the collection tubes from step o) in the centrifuge until the centrifuge is full;
q) upon completion of centrifugation, sequentially retrieving each of the collection tubes from the centrifuge and decanting supernatant;
r) sequentially adding water into each of the collection tubes from q) to resuspend sample and removing each of the sample suspensions to each of a series of sample vials.

Embodiment 13 is the automated plasmid preparation process of embodiment 11 or 12, wherein, after step b), each of the sequence of incubators is programmed to sense the completion of inoculation and start incubation or the multi-axis robot is programmed to signaling each of the sequence of incubators to start incubation.

Embodiment 14 is the automated plasmid preparation process of my one of embodiments 11-13, wherein, after step c) and after step g), the centrifuge is programmed to sense the completion of loading to start centrifugation or the multi-axis robot is programmed to signal the centrifuge to start centrifugation.

Embodiment 15 is the automated plasmid preparation process of any one of embodiments 11-14, wherein, after step i),
if the vacuum manifold is not full,
   I) if the centrifuge from step d) is not empty, repeating steps d)-i);
   II) if the centrifuge from step d) is empty and the one of the sequence of incubators from step c) is not empty, repeating steps c)-i);
   III) if the centrifuge from step d) is empty, the one of the sequence of incubators from step c) is empty, and the next incubator in sequence has sent completion signal, repeating steps c)-i); and
   IV) if the centrifuge from step d) is empty, the one of the sequence of incubators from step c) is empty, and the next incubator in sequence has not sent completion signal, wait until the next incubator in sequence has sent completion signal to repeat steps c)-i).

Embodiment 16 is the automated plasmid preparation process of any one of embodiments 11-15, wherein, after step r), if there are remaining culture bottles from step c), repeating steps c)-r); and if there are no remaining culture bottles from step c), repeating steps a)-r) or ending process.

Embodiment 17 is the automated plasmid preparation process of my one of embodiments 11-16, wherein, after step h), the multi-axis robot is further programmed to place the culture bottles to recycling station for cleaning and reuse.

Embodiment 18 is the automated plasmid preparation process of ay one of embodiments 11-17, wherein, after step k) or 1), the multi-axis robot is further programmed to dry the DNA column by blotting and/or vacuum.

Embodiment 19 is the automated plasmid preparation process of my one of embodiments 11-18, wherein, after step n), the multi-axis robot is further programmed to discard the used DNA columns and remove the column tray.

Embodiment 20 is the automated plasmid preparation process of my one of embodiments 11-19, wherein, after step q), the multi-axis robot is further programmed to wash the pellets remaining in the collection tube with 70% ethanol 1-3 times.

Embodiment 21 is the automated plasmid preparation process of my one of embodiments 11-20, wherein, after step r), the multi-axis robot is further programmed to remove a portion of the sample in each sample vial to microplates for quality control.

1. An automated plasmid preparation module, comprising multiple operation units and at least one multi-axis robot, wherein,
the multiple operation units comprise at least A) a culture and lysis unit for culturing and lysing bacteria transformed with plasmid, B) a purification unit for obtaining purified plasmid DNA, and C) an optional quality control unit; and
the multi-axis robot is programmed to control and operate the multiple operation units.

2. The automated plasmid preparation module of claim 1, further comprising a track, on which the multi-axis robot moves between the multiple operation units.

3. The automated plasmid preparation module of claim 1, wherein, the multi-axis robot comprises a robot arm, a holding means for tube and bottle, a capping/decapping means, a shaking mechanism, one or more liquid dispensing units, and sensor(s) for receiving signals from the multiple operation units.

4. The automated plasmid preparation module of any claim 1, wherein, the A) culture and lysing unit comprises a sequence of incubators with shaking rack(s), one or more centrifuges, and at least one sterilizer, and the B) purification unit comprises one or more vacuum manifolds, one or more centrifuges, one or more column trays each holding a set number of DNA columns, wherein each DNA column has an open upper end, an open lower end, a solid phase capable of plasmid DNA capture, and a filter above the solid phase.

5. An automated plasmid preparation system comprising one or more of the automated integrated plasmid preparation module of claim 1.

6. An automated plasmid preparation process utilizing the automated plasmid preparation system of claim 5, wherein the multi-axis robots are programmed to operate each of the modules simultaneously.

7. An automated plasmid preparation process utilizing the automated plasmid preparation system of claim 5, wherein, the multi-axis robot is programmed to,
A) culture and lyse plasmid DNAs, utilizing the culture and lysing unit; and
B) purify the plasmid DNAs, utilizing the purification unit.

8. The automated plasmid preparation process of claim 7, wherein, in the A) culturing and lysing step, each multi-axis robot is programmed to,
a) receiving a sequence of incubators each holding a set number of empty culture bottles;
b) sequentially for each incubator at a time, filling each of the culture bottles with growth media and inoculating bacteria samples transformed with plasmid;
c) upon completion of incubation of one of the sequence of incubators from step b), retrieving one or more of the culture bottles and loading them into the centrifuge;
d) upon completion of centrifugation, retrieving one culture bottle from the centrifuge and decanting the supernatant;
e) sequentially adding resuspension reagent, lysis reagent, and neutralization reagent into the culture bottle from step d), each addition followed by gentle shaking;
f) placing the culture bottle from step e) in the centrifuge;
g) repeating steps d)-f) until the centrifuge is full.

9. The automated plasmid preparation process of claim 7, wherein, in the B) purifying step, each multi-axis robot is programmed to,
h) retrieving equilibrated DNA columns and after step g) and upon completion of centrifugation, sequentially retrieving each of the culture bottles from the centrifuge and moving the supernatant on top of the filter into each of the equilibrated DNA columns;
i) placing the DNA columns on top of vacuum manifold which is capable to hold a set of DNA columns;
j) applying pressure on the DNA columns and discarding the elute;
k) adding wash buffer into the DNA columns, applying pressure on the DNA columns, and discarding the filter and elute;
l) optionally repeating step k) 1-3 times;
m) for each one of the DNA binding columns, placing one collection bottle underneath thereof;
n) adding elution buffer in each of the DNA columns from step m) and applying pressure on the DNA columns to elute aqueous sample in the collection tubes;
o) adding isopropyl alcohol in each of the collection tubes from step n) followed by vortexing;
p) sequentially placing each of the collection tubes from step o) in the centrifuge until the centrifuge is full;
q) upon completion of centrifugation, sequentially retrieving each of the collection tubes from the centrifuge and decanting supernatant;
r) sequentially adding water into each of the collection tubes from q) to resuspend sample and removing each of the sample suspensions to each of a series of sample vials.

10. The automated plasmid preparation process of claim 8, wherein, after step b), each of the sequence of incubators is programmed to sense the completion of inoculation and start incubation or the multi-axis robot is programmed to signaling each of the sequence of incubators to start incubation.

11. The automated plasmid preparation process of claim 8, wherein, after step c) and after step g), the centrifuge is programmed to sense the completion of loading to start centrifugation or the multi-axis robot is programmed to signal the centrifuge to start centrifugation.

12. The automated plasmid preparation process of claim 8, after step i),
if the vacuum manifold is not full,
i) if the centrifuge from step d) is not empty, repeating steps d)-i);
ii) if the centrifuge from step d) is empty and the one of the sequence of incubators from step c) is not empty, repeating steps c)-i);
iii) if the centrifuge from step d) is empty, the one of the sequence of incubators from step c) is empty, and the next incubator in sequence has sent completion signal, repeating steps c)-i); and
iv) if the centrifuge from step d) is empty, the one of the sequence of incubators from step c) is empty, and the next incubator in sequence has not sent completion signal, wait until the next incubator in sequence has sent completion signal to repeat steps c)-i).

13. The automated plasmid preparation process of claim 6, wherein, after step r), if there are remaining culture bottles from step c), repeating steps c)-r); and if there are no remaining culture bottles from step c), repeating steps a)-r) or ending process.

14. The automated plasmid preparation process of claim 6, wherein, after step h), the multi-axis robot is further programmed to place the culture bottles to recycling station for cleaning and reuse.

15. The automated plasmid preparation process of claim 6, wherein, after step k) or 1), the multi-axis robot is further programmed to dry the DNA column by blotting and/or vacuum.

16. The automated plasmid preparation process of claim 6, wherein, after step n), the multi-axis robot is further programmed to discard the used DNA columns and remove the column tray.

17. The automated plasmid preparation process of claim 6, wherein, after step q), the multi-axis robot is further programmed to wash the pellets remaining in the collection tube with 70% ethanol 1-3 times.

18. The automated plasmid preparation process of claim 6, wherein, after step r), the multi-axis robot is further programmed to remove a portion of the sample in each sample vial to microplates for quality control.
